(19) United States
(12) Patent Application Publication
(10) Pub. No.: US 2005/0107394 A1
(43) Pub. Date: May 19, 2005

(54) Novel use of selective pde5 inhibitors

(76) Inventors: Ardeschir Ghofrani, Giessen (DE); Friedrich Josef Grimminger, Butzbach (DE); Christian Schudt, Konstanz (DE)

Correspondence Address: NATH & ASSOCIATES PLLC, 1030 FIFTEENTH STREET, N.W., SIXTH FLOOR, WASHINGTON, DC, 20005, US

(21) Appl. No.: 10/499,215

(22) PCT Filed: Dec. 14, 2002
(86) PCT No.: PCT/EP02/14279

(30) Foreign Application Priority Data
Dec. 17, 2001 (EP) 011299518
Apr. 26, 2002 (EP) 020095550
Oct. 25, 2002 (EP) 020239364

(51) Int. Cl. A61K 31/519

(52) U.S. Cl. 514/252.16; 514/262.1

(57) ABSTRACT

The invention relates to the novel use of PDE5 inhibitors for the treatment of patients in which a mismatch is present.

### TECHNICAL FIELD

The invention relates to novel use of selective PDE5 inhibitors in the treatment of pulmonary disorders.

### PRIOR ART

In the healthy lung both at rest and during exercise there are always areas of good and poor or absolutely no ventilation existing simultaneously side by side (ventilation inhomogeneity). An as yet unknown mechanism ensures that there is ittle or no perfusion of the capillaries adjacent to alveoli with little or no ventilation. This occurs in order to minimize inefficient perfusion of areas of the lung which are not involved in gas exchange.

Necessary for efficient gas exchange in the lung is a dynamic adaptation of the perfusion conditions to the continual changes in regional ventilation. This coupling is referred to as matching and is determined qualitatively and quantitatively as the V/Q ratio (ventilation/perfusion) by means of the multiple inert gas elimination technique (MIGET).

During bodily exercise, the distribution of ventilation changes (recruitment of new alveoli) and there is increased perfusion of the relevant capillary bed. Conversely, when there is less ventilation due to physiological or pathological processes (airway obstruction), the capillary flow are reduced through vasoconstriction. This process is referred to as “hypoxic vasoconstriction” (Euler-Liljestrand mechanism).

When this adaptation mechanism is impaired (“mismatch”), there may, despite adequate ventilation and normal perfusion of the lungs, be a more or less pronounced collapse of the gas exchange function, which can be compensated only inadequately despite a further increase in ventilation or perfusion. Under these conditions there are regions which are not ventilated but are well perfused (shunting) and those which are well ventilated but not perfused (dead space ventilation), and all intermediate states characterized by deviations from the normal value of V/Q=1. These are, on the one hand, low-V/Q areas (hyperperfusion with little ventilation), and on the other hand high-V/Q areas (hypoperfusion with hyperventilation). The consequence of this mismatch are hypoxaemia (deterioration in gas exchange with decrease in the oxygen content of the patient's blood), wasted perfusion (uneconomical perfusion of unventilated areas) and wasted ventilation (uneconomical ventilation of poorly perfused areas). This leads to a limitation in the patient's performance due to a deficient oxygen supply to the muscles in combination with a “squandering” of cardiorespiratory reserves. The clinical symptoms are a limitation on performance and exercise-dependent or permanent dyspnoea.

In patients with inflammatory and degenerative lung disorders such as, for example, chronic obstructive bronchitis (COPD), bronchial asthma, pulmonary fibroses, emphysema, interstitial pulmonary disorders and pneumonias there is observed to be partial or global respiratory failure. The cause is inadequate adaptation of the intrapulmonary perfusion conditions to the inhomogeneous pattern of the distribution of ventilation. The mismatch derives from the effect of vasoactive (inflammatory) mediators which prevail over the physiological adaptation mechanism. This effect is particularly evident during exercise and when the oxygen demand is increased and it is manifested by dyspnoea (hypoxia) and limitation of performance.

Administration of vasodilators (endothelin antagonists, angiotensin II antagonists, prostacyclin [systemically administered, orally or intravenously], calcium channel blockers) may considerably exacerbate the impairment of the gas exchange function, caused by nonselective vasodilation, especially in the poorly ventilated areas of the lungs, resulting in an increase in mismatch and shunting.

Administration of a vasodilator (especially nitric oxide, NO) by inhalation has a theoretically preferred effect only in the well-ventilated areas of the lungs. However, this requires an efficient inhalation technique which is troublesome for the patient. Additional factors are the systemic effects on absorption through the alveolar epithelium (especially with substances having a long duration of action) and the possible irritation of the bronchial system.

Bronchodilators are intended to reduce airway obstruction which is present. However, in previously damaged lungs they may in fact aggravate further the mismatch, which is the main cause of the reduced performance, through increasing the ventilation in so-called high-V/Q areas and by unwanted systemically vasodilatation (increase in perfusion in low-V/Q areas).

A whole series of PDE5 inhibiting substances (PDE=phosphodiesterase) are known from the prior art and are described as potent and effective substances for the treatment of erectile dysfunction. In addition, EP 1097911 discloses that PDE5-inhibiting substances can be employed for the treatment of pulmonary hypertension and Prasad et al. [Prasad et al. (2000) New England Journal of Medicine 343: 1342] postulate a beneficial role of Sildenafil in primary pulmonary hypertension. EP 758653 discloses that PDE inhibitors are useful for treating bronchitis, chronic asthma, and hypertension.

Grimminger et al. [Grimminger F et al. (2000) Zeitschrift far Kardiologie 89:477] disclose that there are two pharmacological approaches to reduce vascular resistance in patients suffering from chronic pulmonary hypertension: (1) Use of anti-agulatory and fibrinolytic drugs and (2) use of vasodilators with anti-inflammatory and anti-proliferative potency such as prostanoids. Grimminger at al. disclose that the inhalative route of administration is superior because of the pulmonary selectivity and that the decrease in pulmonary-vascular resistance is paralleled by both optimized ventilation-perfusion machting as well as subsequently improved gas exchange. Grimminger et al. also disclose the use of inhaled nitric oxide and aerosolized prostacyclin in ventilated patients with septic lung failure.

Barnes et al. [Barnes P J et al. (1995) Eur. Resp. J. 8:457] describe the involvement of PDE5 in the degradation of cGMP in smooth muscle cells of the airways and vessels.

Kleinsasser A. et al. [Kleinsasser A. et al. (2001) American Journal of Respiratory and Critical Care Medicine 163:339] describes the demonstration that sildenafil modulates the haemodynamics and pulmonary gas exchange in a pig model. However, the skilled person is aware that there are differences between the human and porcine species in relation to pulmonary haemodynamics and gas exchange [Mazzone R W et al. (1981) J.Appl.Physiol 51:739; Woolcock A J et al. (1971) J. Appl. Physiol 30:99; Hogg W et al. (1972) J. Appl. Physiol 33:568; Kurlyama T (1981) J. Appl. Physiol 51:1251; Hedenstiema G et al. (2000) Respir. Physiol. 120:139; Bastacky J et al. (1992) J. Appl. Physiol 73:88]. Thus, pigs lack so-called collateral ventilation. In addition, in pigs there is the phenomenon of pulmonary vascular hyperagility (compared with humans). It is thus clear to the skilled person that results in the pig model cannot be applied directly to humans.

### DESCRIPTION OF THE INVENTION

The object of the present invention is thus to provide a substance which, on oral, intravenous or else inhalational administration, leads on the one hand to the preferred dilatation of vessels in the pulmonary circulation (pulmonary selectivity) and, at the same time, to a redistribution of the blood flow within the lung in favour of the well-ventilated areas

It has now been found, surprisingly, that selective PDE5 inhibitors are suitable for the treatment of patients having the abovementioned mismatch. Administration of selective PDE5 inhibitors leads to dilatation of vessels in the pulmonary circulation and, at the same time, to a redistribution of the blood flow within the lung in favour of the well-ventilated areas. This principle, referred to hereinafter as rematching, leads to an improvement in the gas exchange function both at rest and during physical exercise.

Contrary to the skilled person's expectation, that the vasodilating effect achieved with a PDE5 inhibitor has neither pulmonary or intrapulmonary selectivity, it emerges that there was not only a deterioration but in most cases a significant improvement of pre-existent gas exchange impairments in the treated patients. Selective PDE5 inhibitors are thus suitable as rematching medicament. This improvement in the oxygen supply is not brought about by the well-known general (pulmonary and systemic) vasorelaxation which is typical of PDE5 inhibitors. On the contrary, the improvement in gas exchange derives from PDE5 inhibitors bringing about or enhancing a lung-selective and intrapulmonary-selective vasodilatation in the well-ventilated regions. It is thus possible in patients with a pronounced gas exchange impairment to improve markedly a restricted oxygen supply through administration of selective PDE5-inhibiting substances. In addition, the functional capacity of these patients is significantly improved through a reduction in wasted ventilation and wasted perfusion.

The invention thus relates to the use of PDE5 inhibitors for the treatment of partial and global respiratory failure.

According to the invention, selective PDE5 inhibitors and PDE5 inhibitors are regarded as synonymous.

In connection with this invention, use of PDE5 inhibitors refer to the use of at least one PDE5 inhibitor.

According to this invention, respiratory failure relates to an impairment of oxygen uptake or carbon dioxide release in the lung. Partial respiratory failure according to the invention relates to a fall in the O₂ partial pressure in the blood as a manifestation of the aforementioned impairment of oxygen uptake or carbon dioxide release. According to this invention, global respiratory failure relates to a fall in the O₂ partial pressure in the blood and a rise in the CO₂ partial pressure in the blood as a manifestation of the aforementioned impairment of oxygen uptake or carbon dioxide release.

The invention further relates to the use of PDE5 inhibitors for producing medicaments for the treatment of partial and global respiratory failure.

The invention further relates to the use of PDE5 inhibitors for producing medicaments for the treatment of respiratory failure in patients who have a mismatch of pulmonary ventilation and pulmonary perfusion.

According to this invention, a patient is a human. Preferably, a patient refers to a human in need of medical care or treatment.

The mechanism of the intrapulmonary-selective effect of selective PDE5 inhibitors is based on the inhomogeneity of substrate distribution (cGMP, cyclic guanosine monophosphate) caused by vasodilatation during normal ventilation.

According to this invention, vasodilatation during normal ventilation relates to a local increase in activity of NO synthase in well-ventilated lung areas due to alveolar distension. This results in an increased cGMP synthesis (activation of guanylate cyclase by NO) compared with poorly ventilated lung areas.

It can be stated on the basis of the findings which have been obtained that selective PDE5 inhibitors are able to enhance, in the sense of physiological adaptation of ventilation and perfusion, the necessary vasodilatations specifically in the well-ventilated regions in that they accentuate the physiological inhomogeneity of cGMP distribution in the lung and thus promote rematching. Gas exchange is intensified and the oxygen supply is improved by this mechanism. Selective PDE5 inhibitors thus make selective relaxation of pulmonary vessels possible at the site of adequate ventilation.

A mismatch of pulmonary ventilation and pulmonary perfusion—up to the extremes of dead space ventilation and the shunting—may be caused by various inflammatory and degenerative lung disorders.

This mismatch may be present even at rest but may also appear only under conditions of increased ventilation and perfusion (meaning during exercise) (stress failure of the mismatch).

The invention thus relates to the use of selective PDE5 inhibitors for producing medicaments for the treatment of respiratory failure in patients with an exercise-related mismatch.

The phenomenon of exercise-induced ventilation/perfusion inhomogeneity occurs not only when there are underlying lung disorders, but also during normal aging processes (aging). However, in contrast to inflammatory and degenerative lung disorders, the main feature of age-related mismatch is an increasing rigidity of the pulmonary vessels, resulting in loss of the adaptation-optimizing physiological reflexes (hypoxic vasoconstriction). The mode of action of selective PDE5 inhibitors in these cases derives preferentially from the regionally selective vasodilating effect of the substances and the augmentation of the physiological residual signal (endogenous NO/prostacycline).

The invention further relates to the use of selective PDE5 inhibitors for producing medicaments for the treatment of respiratory failure in patients with an age-related mismatch.

The invention further relates to the use of selective PDE5 inhibitors for producing medicaments for the treatment of respiratory failure in patients with a pathologically caused mismatch.

Patients with a pathologically caused mismatch are patients with a disorder selected from the group consisting of orthopnoea, sleep apnoea and COPD (chronic obstructive pulmonary disease).

The use of selective PDE5-inhibiting substances is suitable specifically in patients with elevated low-V/Q perfusion (V/Q<0.1) to make physiological adaptation (rematching) of pulmonary ventilation and pulmonary perfusion possible through selective vasodilatation at the site of adequate ventilation. According to this invention, an elevated low-V/Q perfusion relates to areas of the lung in which ventilation is low but perfusion is good. A V/Q ratio can be determined in patients with an elevated low-V/Q perfusion through gas exchange measurements by means of MIGET.

The invention further relates to the use of selective PDE5 inhibitors for producing medicaments for the treatment of respiratory failure in patients with a V/Q of <0.1.

The invention additionally relates to the use of selective PDE5 inhibitors in the production of medicaments for the treatment of COPD patients with a predominating bronchitic component (0.001<V/Q<0.1).

COPD patients with a predominanting bronchitic component (called “blue bloaters”) are distinguished by the presence of low-V/Q areas. PDE5 inhibitors contribute to rematching in this subgroup of patients through the predominant vasodilatation in the remaining ventilated areas of the lung.

The invention further relates to the use of selective PDE5 inhibitors in the production of medicaments for the treatment of COPD patients with an emphysematous component. In particular, it relates to the use of selective PDE5 inhibitors in the production of medicaments for the treatment of COPD patients with an emphysematous component of V/Q>10. More particularly preferred, it relates to the use of selective PDE5 inhibitors in the production of medicaments for the treatment of COPD patients with a predominating emphysematous component.

COPD patients with a predominating emphysematous component (called “pink puffers”) are distinguished by the presence of high-V/Q areas and increased dead-space ventilation as the cause of their mismatch. PDE5 inhibitors can contribute to rematching in these patients because of an enhancement of perfusion in the hyperventilated areas (normalization of the V/Q ratio).

The invention additionally relates to the use of selective PDE5 inhibitors in the production of medicaments for the treatment of patients with orthopnoea. Preference is given to those patients suffering from posture-dependent impairments of gas exchange (orthopnoea) with nocturnal desaturation phases.

In a particular group of patients with manifest or latent respiratory failure there is a deterioration in gas exchange on passing from the vertical to the horizontal position (supine position). The change in position results in a redistribution of the ventilation distribution and also of the perfusion distribution, which are only poorly matched in these patients. The limited adaptation capacity means that the matching and correspondingly the O₂ saturation is reduced. This phenomenon is characterized clinically as orthopnoea. The patient develops critical phases of hypoxia, especially during periods of sleep, with the dange of unnoticed undersupply of oxygen, especially to the brain and myocardium. Selective PDE5 inhibitors are able, owing to the rematching effect, to increase the O₂ saturation in these patients and to reduce the risk of secondary organ damage.

The invention further relates to the use of selective PDE5 inhibitors in the production of medicaments for the treatment of patients suffering from sleep apnoea.

According to this invention, sleep apnoea is a nocturnal disturbance of respiratory regulation in which arterial hypoxia develops. These patients differ from other patients in that, owing to failure of the central respiratory drive or owing to anatomically caused peripheral obstruction (tongue versus the upper airways), alveolar ventilation is restricted as alveolar hypoxia is induced. The hypoxic vasoconstriction induced thereby with a subsequent rise in the pulmonary vascular resistance and severe stress on the right heart leads to damage to the myocardium (cor pulmonale) and to the blood vessels (essential hypertension). Administration of conventional vasodilators can certainly dilate the pulmonary vessels and thus reduce the stress on the right heart, but at the cost of a further deterioration in the already impaired gas exchange function through aggravation of the mismatch. Administration of selective PDE5 inhibitors thus makes it possible simultaneously to reduce the pulmonary vascular resistance and to prevent or reduce the mismatch.

The invention further relates to the use of selective PDE5-inhibiting substances in the production of medicaments for the treatment of a therapy-indiced mismatch.

In the treatment of patients with respiratory failure with β2 agonists, theophylline or systemic vasodilators (endothelin antagonists, Ca channel blockers, ACE inhibitors, ATII antagonists, β blockers) there is enhancement of a mismatch which is present. Although the vascular resistance in the lung is reduced on treatment with these medicines, simultaneously the O₂ saturation is reduced. This loss of O₂ saturation increasingly reduces the functional capacity of a patient which is already limited. Consequently, a latent or manifest respiratory failure may be induced in these patients through intake of nonselective vasodilators which is necessary to treat other disorders (therapy-induced mismatch). Selective PDE5 inhibitors are suitable for treating this type of respiratory failure.

Preference is given to uses of selective of PDE5 inhibitors for the treatment of a therapy-induced mismatch on administration of nonselectively vasodilating medicaments, especially nonselectively vasodilating antiobstructive agents.

This invention further relates to the use of selective PDE5 inhibitors for producing medicaments for the treatment of muscular dysfunction caused by perfusion/demand mismatch.

In skeletal muscles (including the respiratory muscle) these is a stress-control adaptation of perfusion to the regional energy demand. Regulation of this “perfusion/demand matching” takes place in analogy to the lung through local release of endogenous vasodilators (especially NO/cGMP). The demand-oriented perfusion is in favour of the stressed muscle groups (muscular selectivity), and within the muscle groups in favour of the specifically stressed fibre types (intramuscular selectivity). The type of stress, duration of stress and level of stress thus determine under physiological conditions the specific perfusion profiles in each case. Various inflammatory disorders (COPD, interstitial lung disorders, infections, vasculitides, degenerative vascular disorders, metabolic disorders), but also the use of nonselective vasoactive medicines for the treatment of the abovementioned disorders, may lead to a perfusion/demand mismatch. The consequence is wasted perfusion of unstressed muscle groups to the detriment of perfusion of stressed muscle groups, with the result of a limitation on muscular performance. PDE5 inhibitors are able to augment the physiological NO/cGMP distribution pattern and thus achieve muscular rematching.

This invention further relates to a medicament preparation comprising at least one selective PDE5 inhibitor and at least one nonselectively vasodilating antiobstructive agent. Such a combination is preferred for the treatment of partial and global respiratory failure. Such a combination is particularly preferred for the treatment of disorders selected from the group consisting of COPD, bronchial asthma, latent pulmonary hypertension associated with underlying lung disorder, emphysema, combined ventilation impairments, chronic left heart failure with pulmonary congestion.

Antiobstructive agents which may induce, for example, endothelin antagonists, Ca channel blockers, ACE inhibitors, ATII antagonists and β blockers. Examples of antiobstructive agents which may be mentioned are endothelin antagonists such as ATRASENTAN, BMS-193884, BOSENTAN, BSF-302146, DARUSENTAN, EDONENTAN, J-104132, SB-209670, SITAXENTAN, TBC-3711, TEZOSENTAN and YM-598, Ca channel blockers such as AMLODIPINE, ARANIDIPINE, BARNIDIPINE, BENCYCLANE, BENIDIPINE, BEPRIDIL, BUFLOMEDIL, CAROVERINE, CILNIDIPINE, CINNARIZINE, DILTIAZEM, DROPRENILAMINE, EFONIDIPINE, FASUDIL, FELODIPINE, FENDILINE, FLUNARIZINE, GALLOPAMIL, ISRADIPINE, LACIDIPINE, LERCANIDIPINE, LIDOFLAZINE, LOMERIZINE, MANIDIPINE, NICARDIPINE, NIFEDIPINE, NILVADIPINE, NIMODIPINE, NISOLDIPINE, NITRENDIPINE, PERHEXILINE, TERODILINE and VERAPAMIL, ACE inhibitors such as ALACEPRIL, BENAZEPRIL, CAPTOPRIL, CERONAPRIL, CILAZAPRIL, DELAPRIL, ENALAPRIL, ENALAPRILAT, FOSINOPRIL, IMIDAPRIL, LISINOPRIL, MOEXIPRIL, PERINDOPRIL, QUINAPRIL, RAMIPRIL, RENTIAPRIL, SPIRAPRIL, TEMOCAPRIL and TRANDOLAPRIL, ATII antagonists such as ABITESARTAN, CL-329167, DA-727, ELISARTAN, EMD-66397, FK-739, HR-720, ICI-D-8731, IRBESARTAN, KRH-594, LR-B/057, MILFASARTAN, OLMESARTAN MEDOXOMIL, POMISARTAN, PRATOSARTAN, RIPISARTAN, SAPRISARTAN, TAK-536, TASOSARTAN, TELMISARTAN, U-96849, VALSARTAN and ZOLASARTAN, and β blockers such as CEBUTOLOL, ALPRENOLOL, AROTINOLOL, ATENOLOL, BEFUNOLOL, BETAXOLOL, BEVANTOLOL, BISOPROLOL, BOPINDOLOL, BUNITROLOL, BUPRANOLOL, CARAZOLOL, CARTEOLOL, CARVEDILOL, CELIPROLOL, DILEVALOL, ESMOLOL, LABETALOL, LEVOBUNOLOL, MEPINDOLOL, METIPRANOLOL, METOPROLOL, MOPROLOL, NADOLOL, NEBIVOLOL, NIPRADILOL, OXPRENOLOL, PENBUTOLOL, PINDOLOL, PRACTOLOL, PROPRANOLOL, SOTALOL, TALINOLOL, TERTATOLOL, TILISOLOL, TIMOLOL, TOLIPROLOL and XAMOTEROL.

Nonselectively vasodilating antiobstructive agents are used in medicaments for the treatment of obstructive ventilation impairment. Administration of such antiobstructive agents may considerably exacerbate the disturbance of gas exchange function, caused by a nonselectve vasodilatation—especially in the poorly ventilated lung areas—which may lead to an increase in mismatch and shunting. PDE5 inhibitors are able to show their selective effect also in combination with nonselectively vasodilating antiobstructive agents and, through their selective effect, compensate the mismatch caused by the nonselectively vasodilating antiobstructive agents. Nonselectively vasodilating antiobstructive agents and selective PDE5 inhibitors can be administered in a fixed combination. It is likewise possible to administer nonselectively vasodilating antiobstructive agents and selective PDE5 inhibitors as free combination—singly—in which case administration can take place in immediate succession or at a relatively large time interval. According to this invention, a relatively large time interval relates to a time interval of up to a maximum of 24 hours.

Substances which may be included among PDE5 inhibitors and selective PDE5 inhibitors for example are those described and claimed in the following patent applications and patents: WO 9626940, WO 9632379, EP 0985671, WO 9806722, WO 0012504, EP 0667345, EP 0579496, WO 9964004, WO 9605176, WO 9307124, WO 9900373, WO 9519978, WO 9419351, WO 9119717, EP 0463756, EP 0293063, WO 0012503, WO9838168, WO 9924433, DE 3142982 and U.S. Pat. No. 5,294,612.

Compounds which may be mentioned as examples of PDE5 inhibitors and selective PDE5 inhibitors are 3-ethyl-8-[2-(4-morpholinylmethyl)benzylamino]-2,3-dihydro-1H-imidazo[4,5-g]quinazoline-2-thione, 1-(2-chlorobenzyl)-3-isobutyryl-2-propylindole-6-carboxamide, 9-bromo-2-(3-hydroxypropoxy)-5-(3-pyridylmethyl)-4H-pyrido[3,2,1-[k]-carbazol-4-one, 4-(1,3-benzodioxol-5-ylmethylamino)-2-(1-imidazolyl)-6-methylthieno [2,3-pyrimidine, 6-(2-isopropyl-4,5,6,7-terahydropyrazolo[1,5-a]pyridin-3-yl)-5-methyl)-5-methyl-2, 3,4,5-tetrahydropyridazin-3-one, 5-(4-methylbenzyl)-3-(1-methyl-4-phenylbutyl)-3,6-dihydro-[1,2,3]triazolo[4,5-d]pyrimidin-7-one, 3-(1-methyl-4-phenylbutyl)-5-pyridin-4-ylmethyl-3,6-dihydro-[1,2,3]triazolo[4,5-d]pyrimidin-7-one, 5-(4-bromobenzyl)-3-(1-methyl-4-phenylbutyl)-3,6-dihydro-[1,2,3]triazolo[4,5-d]pyrimidin-7-one, 5-benzyl-3-(1-methyl-4-phenylbutyl)-3,6-dihydro-[1,2,3]triazolo[4,5-d]pyrimidin-7-one, 5-(3,4-dimethoxybenzyl)-3-(1-methyl-4-phenylbutyl)-3,6-dihydro-[1,2,3]triazolo-[4,5-d]pyrimidin-7-one, 5-(3,4-dichlorobenzyl)-3-(1-methyl-4-phenylbutyl)-3,6-dihydro-[1,2,3]triazolo[4,5-d]pyrimidin-7-one, 5-biphenyl-4-ylmethyl-3-(1-methyl-4-phenylbutyl)-3,6-dihydro-[1,2,3]triazolo[4,5-d]pyrimidin-7-one, 5-(4-aminobenzyl-3-(1-methyl-4-phenylbutyl)-3,6-dihydro-[1,2,3]triazolo[4,5-d]pyrimidin-7-one, 5-(hydroxyphenylmethyl)-3-(1-methyl-4-phenylbutyl)-3,6-dihydro-[1,2,3]triazolo-[4,5-d]pyrimidin-7-one, 5-benzo[1,3]dioxol-5-ylmethyl-3-[1-methyl-4-phenylbutyl]-3,6-dihydro-[1,2,3]triazolo[4,5-d]pyrimidin-7-one, N-4-[3-(1-methyl-4-phenylbutyl)-7-oxo-6,7-dihydro-3H-[1,2,3]triazolo-[4,5-d]pyrimidin-5-ylmethyl]phenylacetamide, 5-benzoyl-3-(1-methyl-4-phenylbutyl)-3,6-dihydro-[1,2,3]triazolo[4,5-d]-pyrimidin-7-one, 3-(1-methyl-4-phenylbutyl)-5-[4-(morpholine-4-sulphinyl) benzyl]-3,6-dihydro[1,2,3]triazolo[4,5-d]pyrimidin-7-one, 3-(1-methyl-4-phenylbutyl)-5-[3-(morpholine-4-sulphonyl) benzyl]-3,6-dihydro[1,2,3]triazolo[4,5-d]pyrimidin-7-one, N-methyl-4-[3-(1-methyl-4-phenylbutyl)-7-oxo-6,7-dihydro-3H-[1,2,3]-triazolo-[4,5-d]pyrimidin-5-ylmethyl]-benzenesulphonamide, N-(2-dimethylaminoethyl)-4-[3-(1-methyl-4-phenylbutyl)-7-oxo-6,7-dihydro-3H-[1,2,3]triazolo[4,5-d]pyrimidin-5-ylmethyl]benzenesulphonamide, N-(2-hydroxyethyl)-4-[3-(1-methyl-4-phenylbutyl)-7-oxo-6,7-dihydro-3H-[1,2,3]triazolo[4,5-d]pyrimidin-5-ylmethyl]benzenesulphonamide, ethyl 1-[3-[3-(1-methyl-4-phenylbutyl)-7-oxo-6,7-dihydro-3H-[1,2,3]-triazolo-[4,5-d]pyrimidin-5-ylmethyl]benzenesulphonyl]piperidinecarboxylate, 3(1-methyl-4-phenylbutyl)-5-[4-(4-methylpiperazin-1-sulphonyl) benzyl]-3,6-dihydro-[1,2,3]triazolo[4,5d]pyrimidin-7-one, 5-benzo[1,3]dioxol-5-ylmethyl-3-[1-ethyl-heptyl]-3,6-dihydro-[1,2,3]-triazolo[4,5-d]pyrimidin-7-one, 3-[1-(1-hydroxyethyl)-4-phenylbutyl]-5-[4-(morpholine-4-sulphonyl) benzyl]-3,6-dihydro-[1,2,3]triazolo[4,5-d]pyrimidin-7-one, 5-[6-fluoro-1-(phenylmethyl)-1H-indazol-3-yl]-2-furanmethanol, 1-benzyl-6-fluoro-3-[5-(hydroxymethyl)furan-2-yl]-1H-indazole, 2-(1H-imidazol-1-yl)-6-methoxy-4-(2-methoxyethylamino)quinazoline, 1-[[3-dihydro-8-oxo-1H-imidazo[4,5-g]quinazolin-6-yl)-4-propoxyphenyl]sulphonyl]-4-methylpiperazine, 4-(3-chloro-4-methoxybenzylamino)-1-(4-hydroxypiperidin-1-yl) phthalazine-6-carbonitrile, 1-[6-chloro-4-(3,4-methylendioxybenzylamino)quinazolin-2-yl]piperidin-4-carboxylic acid, (6R, 12aR)-6-(1,3-benzodioxol-5-yl)-2-methyl-1, 2,3,4,6,7,12,12a-octa-hydropyrazino[2′,1′:6,1]pyrido[3,4-b]indole-1,4-dione (tadalafil), (6R,12aR)-2,3,6,7,12,12a-hexahydro-2-methyl-6-(3,4-methylenedioxyphenyl)-pyrazino-[2′,1′:6,1]pyrido[3,4-b]indole-1,4-dione, 4-ethoxy-2-phenylcycloheptylimidazole, (6-bromo-3-methoxymethylimidazo [1,2-a]pyrazin-8-yl)methylamine, 8-[(phenylmethyl)thio]4-(1-morpholinyl)-2-(1-piperazinyl) pyrimidino[4,5-d]pyrimidine, (+)-cis-5-methyl-2-[4-(trifluoromethyl)benzyl]-3,4,5,6a,7,8,9-octahydrocyclopent [4,5]imidazo[2,1-b]purin-4-one, cis-2-hexyl-5-methyl-3,4,5,6a,7,8,9,9a-octahydrocyclopent [4,5imidazo[2,1-b]purin-4-one, 5-[2-ethoxy-5-(4-methyl-1-piperazinyl-sulphonyl) phenyl]-1-methyl-3-n-propyl-1,6-dihydro-7H-pyrazolo[4,3-d]pyrimidin-7-one (slidenafil), 1-[[3-(6,7-dihydro-1-methyl-7-oxo-3-propyl-1H-pyrazolo[4,3-d]pyrimidin-5-yl)-4-ethoxyphenyl]sulfonyl]-4-methylpiperazine, 2-(2-propoxyphenyl)purin-6(1H)-one, 2-(2-propoxyphenyl)-1,7-dihydro-5H-purin-6-one, methyl 2-(2-methylpyridin-4-ylmethyl)-1-oxo-8-(2-pyrimidinylmethoxy)-4-(3,4,5-trimethoxyphenyl)-1,2-dihydro-[2,7]naphthyridin-3-carboxylate, methyl 2-(4-aminophenyl)-1-oxo-7-(2-pyridinylmethoxy)-4-(3, 4,5-trimethoxyphenyl)-1,2-dihydroisoquinoline-3-carboxylate, 2-[2-ethoxy-5-(4-ethylpiperazin-1-ylsulfonyl) phenyl]-5-methyl-7-propylimidazo[5,1-f][1,2,4]triazin-4(3H)-one (vardenafil), 3,4-dihydro-6-[4-(3,4-dimethoxybenzoyl)-1-piperazinyl]-2-(1H)-quinolinone (vesnarinone), 1-cyclopentyl-3-methyl-6-(4-pyridyl) pyrazolo[3,4-d]pyrimidin-4(5H)-one, 1-cyclopentyl-6-(3-ethoxy-4-pyridinyl)-3-ethyl-1,7-dihydro-4H-pyrazolo[3,4-d]-pyrimidin-4-one, 6-o-propoxyphenyl-8-azapurin-6-one, 3,6-dihydro-5-(o-propoxyphenyl)-7H-v-triazolo[4,5-d]pyrimidin-7-one and 4-methyl-5-(4-pyridinyl)thiazole-2-carboxamide and the pharmacologically acceptable salts of these compounds.

PDE5 inhibitors and selective PDE5 inhibitors which are particularly preferred are selected from the group consisting of tadalafil, sildenafil, vardenafil and vesnarinone and the pharmacologically acceptable salts of these compounds.

Suitable salts are—depending on the substitution and depending on the basic structure—in particular all acid addition salts or else salts with bases. Particular mention may be made of the pharmacologically acceptable salts of the inorganic and organic acids normally used in pharmaceutical technology. Suitable as such are water-soluble and water-insoluble acid addition salts with acids such as, for example, hydrochloric acid, hydrobromic acid, phosphoric acid, nitric acid, sulphuric acid, acetic acid, citric acid, D-gluconic acid, benzoic acid, 2-(4-hydroxybenzoyl)benzoic acid, butyric acid, sulphosalicylic acid, maleic acid, lauric acid, malic acid, fumaric acid, succinic acid, oxalic acid, tartaric acid, embonic acid, stearic acid, toluenesulphonic acid, methanesulphonic acid or 3-hydroxy-2-naphthoic acid, the acids being employed in the preparation of salts—depending on whether the acid is monobasic or polybasic and depending on which salt is desired—in the equimolar ratio of amounts or one differing therefrom. Particular mention should also be made of the pharmacologically acceptable salts of the inorganic and organic bases normally used in pharmaceutical technology. Suitable as such are water-soluble and water-insoluble salts with bases such as, for example, sodium hydroxide solution, potassium hydroxide solution or ammonia.

In the use according to the invention of PDE5 inhibitors or selective PDE5 inhibitors for producing the aforementioned medicaments and in the pharmaceutical preparations according to the invention, the PDE5 inhibitors or selective PDE5 inhibitors (=the active ingredients) are processed with suitable pharmaceutical excipients or carriers to tablets, coated tablets, capsules, suppositories, plasters (e.g. as transdermal therapeutic system=TTS), emulsions, suspensions or solutions, with the active ingredient content advantageously being between 0.1 and 95%, and it being possible by appropriate choice of the excipients and carriers to obtain a pharmaceutical dosage form (e.g. a slow-release form or an enteric form) which is exactly adapted to the active ingredient and/or to the desired onset of action.

Excipients and carriers suitable for the desired pharmaceutical formulations are familiar to the skilled person on the basis of his expert knowledge. Besides solvents, gel formers, suppository bases, tablet excipients and other active ingredient carriers it is possible to use, for example, antioxidants, dispersants, emulsifiers, antifoams, masking flavours, preservatives, solubilizers, colours or, in particular, permeation promoters and complexing agents (e.g. cyclodextrins).

The active ingredient can be administered orally, by inhalation, percutaneously, transdermally or intravenously.

It has generally proved advantageous in human medicine to administer the active ingredient in the case of oral administration in a daily dose of about 0.02 to about 4 mg, in particular 0.1 to 2 mg per kg of body weight, where appropriate in the form of a plurality of, preferably 1 to 3, individual doses to achieve the desired result, with gradually increasing and decreasing dosage possibly being advantageous. On parenteral treatment it is possible to use similar or (especially on intravenous administration of the active ingredient) usually lower dosages.

The skilled person is aware that the optimal dose of an active ingredient may vary depending on the body weight, the age and the general condition of the patient, and on his response to the active ingredient.

Every skilled person is easily able to establish on the basis of his expert knowledge the optimal dosage and mode of administration of the active ingredient necessary in each case.

The invention further relates to a commercial product consisting of a conventional secondary packaging, of a primary packaging containing the medicament (for example an ampoule or a blister) and, if desired, a package insert, where the medicament is used for the treatment of partial and global respiratory failure, the suitability of the medicament for the treatment of partial and global respiratory failure is indicated on the secondary packaging and/or on the package insert of the commercial product, and the medicament comprises a PDE5 inhibitor. The secondary packaging, the medicament-containing primary packaging and the package insert otherwise correspond to that which the skilled person would regard as standard for medicaments of this type.

The invention further relates to a ready-to-use medicament comprising a PDE5 inhibitor and an indication that this medicament can be employed for the treatment of partial and global respiratory failure.

The invention further relates to a method of treating partial and global respiratory failure in a human in need thereof comprising the step of administering to said human a therapeutically effective amount of a PDE5 inhibitor.

According to this invention, a therapeutically effective amount of a PDE5 inhibitor refers to the pharmacologically tolerable amount of the PDE5 inhibitor sufficient, either as a single dose or as a result of multiple doses, to decrease the mismatch of pulmonary ventilation and pulmonary perfusion, or to reduce wasted perfusion and wasted ventilation.

The invention further relates to a method of treating respiratory failure in a human showing a mismatch of pulmonary ventilation and pulmonary perfusion comprising the steps of administration to said human in need a therapeutically effective amount of a selective PDE5 inhibitor. In particular, the human in need having a mismatch of V/Q<0.1 are preferred.

The invention further relates to a method of treating respiratory failure in a human showing an exercise-dependent mismatch of pulmonary ventilation and pulmonary perfusion comprising the steps of administration to said human in need a therapeutically effective amount of a selective PDE5 inhibitor.

The invention further relates to a method of treating respiratory failure in a human showing an age-related mismatch of pulmonary ventilation and pulmonary perfusion comprising the steps of administration to said human in need a therapeutically effective amount of a selective PDE5 inhibitor. In particular, the human in need having a mismatch of V/Q<0.1 are preferred.

The invention further relates to a method of treating respiratory failure in a human showing pathologically caused mismatch of pulmonary ventilation and pulmonary perfusion comprising the steps of administration to said human in need a therapeutically effective amount of a selective PDE5 inhibitor. In particular, the human in need having a mismatch of V/Q<0.1 are preferred.

The invention further relates to a method of treating respiratory failure in a COPD patient with a predominant bronchitis component showing a mismatch of pulmonary ventilation and pulmonary perfusion comprising the steps of administration to said human in need a therapeutically effective amount of a selective PDE5 inhibitor. In particular, a COPD patient having a mismatch of V/Q<0.1 are preferred.

The invention further relates to a method of treating respiratory failure in a COPD patent with an emphysematous component showing a mismatch of pulmonary ventilation and pulmonary perfusion comprising the steps of administration to said human in need a therapeutically effective amount of a selective PDE5 inhibitor. In particular, a COPD patient having a mismatch of V/Q>10 is preferred.

The invention further relates to a method of treating orthopnoea in a human showing a mismatch of pulmonary ventilation and pulmonary perfusion comprising the step of administering to said human a therapeutically effective amount of a PDE5 inhibitor.

The invention further relates to a method of treating sleep apnoea in a human showing a mismatch of pulmonary ventilation and pulmonary perfusion comprising the step of administering to said human a therapeutically effective amount of a PDE5 inhibitor.

The invention further relates to a method of treating respiratory failure in a human showing a therapy-induced mismatch of pulmonary ventilation and pulmonary perfusion comprising the steps of administration to said human in need a therapeutically effective amount of a selective PDE5 inhibitor.

The invention further relates to a method of treating respiratory failure in a human showing a mismatch of pulmonary ventilation and pulmonary perfusion caused by administration of nonselectively vasodilating medicaments, the method comprises the steps of administration to said human in need a therapeutically effective amount of a selective PDE5 inhibitor. In particular, the method is preferred, wherein the nonselectively vasodilating medicament is a nonselectively vasodilating antiobstructive agent. The method is particularly preferred, wherein the nonselectively vasodilating antiobstructive agent is selected from the group consisting of endothelin antagonist, Ca channel blocker, ACE inhibitor, ATII antagonist and β blocker.

The invention further relates to a method of treating muscular dysfunction in a human showing a perfusion/demand mismatch comprising the step of administering to said human a therapeutically effective amount of a PDE5 inhibitor.

Further advantages and embodiments of the invention are described below and are evident from the examples and the appended drawings.

### DESCRIPTION OF THE FIGURES

FIG. 1: Result of determination of shunting with the aid of the model of bleomycin-induced pulmonary fibrosis in rabbits. The measurements by the inert gas exchange method (MIGET) [Wagner et al., J Appl Physiol. 1974;36:588-99] reveal that the shunting was increased by 15% in this model, compared with the untreated control. Systemically administered PGI [6 ng/kg body weight/min] (PGI, prostacyclin) increased the shunting to about 30%. Exogenous inhaled NO [20 parts per million (ppm)] by contrast reduced the shunting to 9%. Shunting was reduced to 6% by oral administration of the PDE5 inhibitor sidenafil [1 mg/kg body weight].

FIG. 2: Result of determination of the oxygenation index (arterial oxygen partial pressure/fraction of inspired oxygen [PaO₂/FiO₂]) measured in the model of bleomycin-induced pulmonary fibrosis in rabbits. Whereas systemic PGI (6 ng/kg body weight/min] (PGI, prostacyclin) reduces the oxygenation index by 60% compared with the control (190), the oxygenation index was markedly raised by inhaled NO [20 ppm] by 28% and sildenafil (oral) [1 mg/kg body weight] by 31%.

FIG. 3: Result of determination of the low V/Q perfusion measured by the inert gas exchange method (MIGET) [Wagner et al. J Appl Physiol. 1974;36:588-99] from 7 patients with chronic thromboembolism and displaying secondary PHT (pulmonary hypertension). Compared with the control group (3.25%), the shunting was increased with PGI (i.v.) [6 ng/kg bodyweight/min] to 19%, with NO (inhaled) [20 ppm] to 5.3% and with sildenafil (oral) [50 mg] to 5.3%.

FIG. 4: Result of measurement of the arterial oxygen partial pressure (PaO₂) on 7 patients with chronic thromboembolism and displaying secondary PHT (pulmonary hypertension). The arterial oxygen partial pressure was improved by NO (inhaled) [20 ppm] and sildenafil (oral) [50 mg] by respectively 1.8% and 7.6%, whereas the oxygen saturation fell by 13% after administration of PGI (intravenous). In the same experiment, the vascular resistance was measured and determined as delta PVR by a right cardiac catheterization. The vascular resistance was reduced respectively by 25%, 19% and 21% after administration of PGI (intravenous) [6 ng/kg bodyweight/min], NO (inhaled) [20 ppm] and sildenafil (oral) [50 mg].

FIG. 5: Result of determination of the shunting on 7 patients with ILD (interstitial lung disease) displaying secondary PHT. The measurement took place by the inert gas exchange method (MIGET) [Wagner et al. J Appl Physiol. 1974;36:588-99]. The shunting was reduced to 5% and 4.8%, respectively, by NO (inhaled) [20 ppm] and sildenafil (oral) [50 mg]. The shunting was increased to 18% after administration of PGI (prostacyclin, intravenous) 16 ng/kg bodyweight/min].

FIG. 6: Result of determination of the arterial oxygen partial pressure (PaO₂) on 7 patients with ILD displaying secondary PHT (pulmonary hypertension) measured as delta PaO2. Whereas the arterial oxygen partial pressure was increased by 4.8% and 13%, respectively, by NO (inhaled) [20 ppm] and sildenafil (oral) [50 mg], the oxygen saturation was reduced by 12.5% in patients after administration of PGI (prostacyclin, intravenous) [6 ng/kg bodyweight/min].

FIG. 7: Result of the 6-minutes walking test measured on 4 patients with COPD (chronic obstructive pulmonary disease). The change in the 6-minutes walking distance on administration of 75 mg of sildenafil (oral) each day for a period of 6 months revealed an improvement respectively of 41%, 45%, 74% and 150% for the 4 patients compared with the starting point before treatment with sildenafil (0 months).

FIG. 8: Result of determination of the arterial oxygen saturation on 4 patients with COPD (chronic obstructive pulmonary disease) treated with sildenafil (75 mg/day) (oral) measured at rest over a period of 6 months. The arterial oxygen saturation in the patient improved respectively by 2%, 4%, 5% and 6%, compared with the saturation at the start of the series of measurements (time: 0 months).

### EXAMPLES

It was surprisingly found in experiments on the isolated perfused lungs that there is oxygen- and ventilation-dependent synthesis of NO in the lung. It was shown in experimental pulmonary fibrosis on whole animals, in patients with chronic persistent thromboembolism and in patients with interstitial lung disease that the vascular resistance decreases and, at the same time, the O₂ saturation is improved on use of the selective PDE5 inhibitor sildenafil (in contrast to PGI₂).

The effect of selective PDE5-inhibiting substances is confined to the area of NO synthesis. A selective PDE5 inhibitor thus achieves its selective vasodilating effect which differs from PGI (“intrapulmonary selectivity ) through enhancing the local NO effect.

The hypothesis that PDE5 inhibitors, in contrast to other vasodilators, improve matching and, correspondingly, increase the O₂ saturation, and thus act as rematching drug, is proved experimentally and clinically by the following results of studies on an animal model and on patients with interstitial lung diseases.

### Example 1

### Isolated Perfused Lung

The isolated, ventilated rabbit lung with bloodless perfusion is an established organ model. Removal of the lung from the integrated organ system makes It possible for the experimental situation to be free of humoral, central and metabolic influences from the body for investigating the complete, isolated, but intact organ. The ex vivo experimental mode used permits continuous recording of measurements of biophysical parameters such as the pulmonary arterial pressure, the ventilation pressure and the lung weight. Modification of the basic design additionally made alveolar deposition of substances possible through nebulization in the present study.

New Zealand White crossbred rabbits of both sexes weighing between 2.6 and 2.8 kg were used to carry out this series of experiments. A marginal ear vein was punctured for injection of the necessary substances. The animals were then sedated with a mixture of ketamine (Ketanest®) and xylazine (Rompun®) (2/3 ratio of amounts) without suppressing spontaneous breathing and anticoagulated with 1 000 I.U. of heparin per kg/bodyweight. To eliminate sensitivity for the subsequent tracheotomy, a weal was raised in the skin with 10 ml of 0.2% Xylocaine®. The trachea was exposed by careful layered dissection and could then be intubated with a metal cannula through a tracheotomy. Positive pressure ventilation with ambient air was then carried out by the attached ventilation pump with a tidal volume of 30 ml, a respiratory rate of 30/min and an end-expiratory pressure of 0 cm H₂O. Following the start of mechanical ventilation, anaesthesia was made more profound with Ketanest®/Rompun® until analgesia and relaxation were complete.

After dissection of the aorta and the pulmonary artery, about 4% CO₂ was added to the ventilation with ambient air. Immediately after this, an incision was performed at the level of the outflow tract of the right ventricle, and the catheter (internal diameter 3 mm) filled with 3-4° C. cooled perfusion medium was introduced into the pulmonary artery. Perfusion was started with 1 0 mlymin. To avoid pressure stress on the pulmonary circulation, immediately thereafter the apex of the heart was opened. The heart-lung specimen was removed after mobilization of the trachea from the posterior wall of the thorax. Finally, the oesophagus and inferior vena cava and remaining strands of connective tissue were severed. To complete the artificial circulation, a catheter was introduced into the left ventricle and fixed by an intramural purse-string suture. The left auricular appendage was, as a possible interfering fluid reservoir, ligated near to the ventricle wall. The dissection was all carried out in a period not exceeding 30 minutes with continuous ventilation and perfusion. The lung was perfused with pulsatile flow from a peristaltic tubing pump. Inflow took place through the catheter which had already been introduced and fixed in the pulmonary artery during dissection. After passing through the pulmonary circulation, venous outflow of the perfusion medium was possible through the tube fixed in the left ventricle. The perfusate flowing out was returned to the reservoir via a ladder-like cascade system. This cascade system made it possible to vary the hydrostatic pressure on the pulmonary vascular system between 0 and 10 cm H₂O (reference point was the hilum of the lung) by closing individual rungs (venous pressure challenge).

The heart-lung package was suspended freely on an electronic weighing cell in a gas tight equilibration vessel for continuous recording of the weight. The perfusate containers consisted of double-walled glass; temperature-control fluid flowed through them from a thermal pump, which made it possible to control the temperature of the perfusate vessels and thus to control the temperature of the perfusate. It is possible to change from ambient air ventilation to hypoxic respiratory gas (FiO₂ 0.03) by means of a selector switch. Simultaneously, the NO release are measured in the exhaled air and in the circulating perfusate. The influence of alveolar distension on NO synthesis and release is found by changing the ventilation pressures (in particular inspiratory pressure and end-expiratory pressure) (PEEP)).

The NO release is influenced by the distension of the alveoli and thus serves as a mediator of ventilation and distension of the alveoli. Consequently, NO synthesis in the lung is controlled by the two parameters of O₂ content and alveolar ventilation. Hypoxia reduces NO synthesis and there is a “stretch-induced” increase in NO release due to alveolar distension. These two mechanisms guarantee, in view of the inhomogeneous ventilation distribution of the lung under normal conditions, that perfusion takes place only where ventilation is good at the same time (“normoxic ventilation”). The increased NO concentration increases the guanylate cyclase activity in the smooth muscle cells of the vessel wall, and smooth muscle cells are relaxed by the resulting CGMP. The vessel cross section (Q) and ventilation (V) are thus directly coupled via NO synthesis and guarantee an optimal V/Q quotients (matching).

### Example 2

### Effect of Sildenafil on Bleomycin-induced Pulmonary Fibrosis

Healthy rabbits of both sexes were pretreated orally with a gyrase inhibitor (Baytril®) for one week. Ten animals pretreated in this way were not treated with bleomyin and served as control, and, on the day of exposure, the others were anaesthetized with a Ketanest®/Rompun® mixture, intubated intratracheally and ventilated mechanically. An ultrasonic nebulizer (MMAD 2.5 μm) was used to administer by inhalation exactly 1.8 U/kg of bodyweight of bleomycin under volume-controlled ventilation. After 4, 8, 16, 24, 32 and 64 days (in each case n≧5) post exposure, the animals were again anaesthetized, provided with an arterial access (right carotid artery) and underwent bodyweight-adapted ventilation via a tracheostomy in a volume-controlled method. The arterial pO₂ and pCO₂, and the static compliance of the lung were measured (by recording the intrathoracic pressure and with slow inflation/deflation manoeuvres). Subsequently, the lungs of these animals were dissected and perfused with a Krebs Hensefeit buffer. Under these conditions, the capillary filtraton coefficient (cfc) was then found from the weight gain of the organ after increasing the pulmonary venous pressure by 7.5 mmHg, and the peak ventilation pressure was found. After completion of these measurements, the left main bronchus was ligated in the end-inspiratory position and a bronchoalveolar lavage (BAL) was performed on the right lung. Subsequently, the large vessels and airways of the right lung were dissected off and the organ was homogenized. The left lung was perfusion-fixed with 4% formalin solution while maintaining a pressure gradient of 25 cm H₂O and was then stored in 4% formalin until embedded. Firstly, the cells were removed from the BAL, counted and differentiated via a Papenheim stain. The cell-free BAL supernatant was then aliquoted and submitted to further analysis of the surfactant and coagulation properties and a determination of the matrix metallo proteinases (MMPs) and their inhibitors (TIMPs) and of soluble collagen. Bronchoalveolar deposition of 1.8 U/kg of bodyweight of bleomycin led initially to development of an ARDS-like event, with a massive restriction of gas exchange (paO₂/FiO₂ of >500 mmHg in the controls reduced to ˜110 mmHg on day 4), ground-glass opacities over all sections of the lung in the HRCT and an increase of about 5-fold in the capillary filtration coefficient (cfc). In the later phase of bleomycin-induced lung damage there was then development of pronounced fibrosis which could be confirmed on the basis of the increase in soluble collagen in the BAL and the hydroxyproline in the tissue, on the basis of the histological specimens and of the HRCT findings. Thus, the concentration of soluble collagen in the BAL increased from 1.1±0.4 μg/ml in the controls to a maximum of 38.3±12.5 μg/ml on day 16 after bleomycin administration and was still distinctly increased even after 64 days, at 7.0±2.2 μg/ml. The hydroxyproline content of the tissue was approximately doubled from day 16 onwards and showed a negligible reduction subsequently. 32 days after exposure, the HRCT revealed a pronounced reticular and homogeneous marking pattern of the lungs. Consistent with this, a pronounced increase in the extracellular matrix and an alveolar and also interstitial ingress of fibroblasts was observable in the histological sections. Besides the homogeneously distributed zones of fibrosis there were also thin hyperdistended sections of lung with a honeycomb appearance.

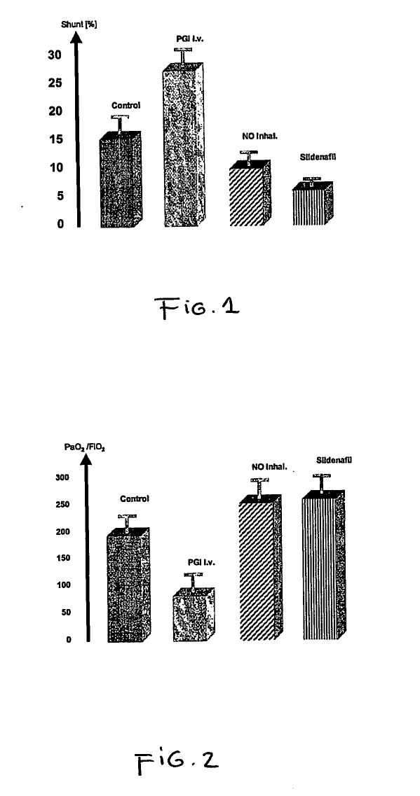

As depicted in FIG. 1, measurements using the inert gas exchange method revealed that shunting was increased by 15% compared with the untreated control in the model of bleomycin-induced pulmonary fibrosis. Systemically administered PGI (vasodilatator) increased the shunting to about 30%. Exogenous inhaled NO by contrast reduced the shunting. The PDE5 inhibitor sidenafil given orally reduced the shunting even more than NO. The data for O2 saturation (FIG. 2) correspond directly. Whereas systemic PGI reduced O₂ saturation, there were marked increases in O₂ saturation with inhaled NO and sildenafil (oral).

Consequently, systemically administered vasodilators do not show intrapulmonary selectivity and enhance perfusion even where there is little or absolutely no ventilation. By contrast, vasodilators administered by inhalation dilate only where there is ventilation and thus show “intrapulmonary selectvity”—the shunting is reduced. PDE5 inhibitors are administered orally and surprisingly show “intrapulmonary selectivity”. Sildenafil differs from the normal vasodilator in reducing shunting.

### Example 3

### Sildenafil in Patients with Chronic Thromboembolism

7 patients with CTEPH underwent a Swan-Ganz catheter investigation with measurement of the ventilation/perfusion (V/Q) distribution (using the multiple inert gas elimination technique (MIGET)). After determination of the baseline parameters (haemodynamics and gas exchange), all the patients initially inhaled 20 ppm NO, followed by a second baseline period (10-15 min), and then PGI was infused (6 ng/kg bodyweight/min), again followed by a second baseline period (10-15 min) and then an oral dose of 50 mg of sildenafil was given (120-150 min follow-up). 3 of the 7 patients were controlled by continuous nasal oxygen therapy in order to reach an arterial oxygenation of >88%. The following parameters were measured under baseline conditions: mean pulmonary arterial pressure (mPAP) 52.1 +/−3.3 mmHg, cardiac index (CI) 2.2+/−0.1 |* min⁻¹*m⁻², pulmonary vascular resistance index (PVRI) 1703.8+/−129.5 dyn*s*cm⁻⁶*m², arterial pO2 72.5+/−3.3 mmHg and mixed venous oxygen saturation (SvO₂) of 63.4+/−2.2%. MIGET demonstrated a V/Q distribution disturbance in the middle V/Q areas (broad distribution of perfusions), a low blood flow through shunt areas ((2.30+/−0.75%) and regions with poor ventilation (low V/Q areas, 3.25+/−1.84%), and a large dead-space ventilation. Administration of NO, PGI and sildenafil led in each case to a marked reduction in the pulmonary vascular resistance. Whereas NO and sildenafil left the ventilation/perfusion distribution virtually unchanged, on PGI infusion there was a considerable increase in the low-V/Q perfusion (to 19%), resulting in a decrease in the arterial oxygen partial pressure during PGI infusion by 13% compared with the control investigation. The perfusion of normally distributed V/Q areas remained virtually unchanged.

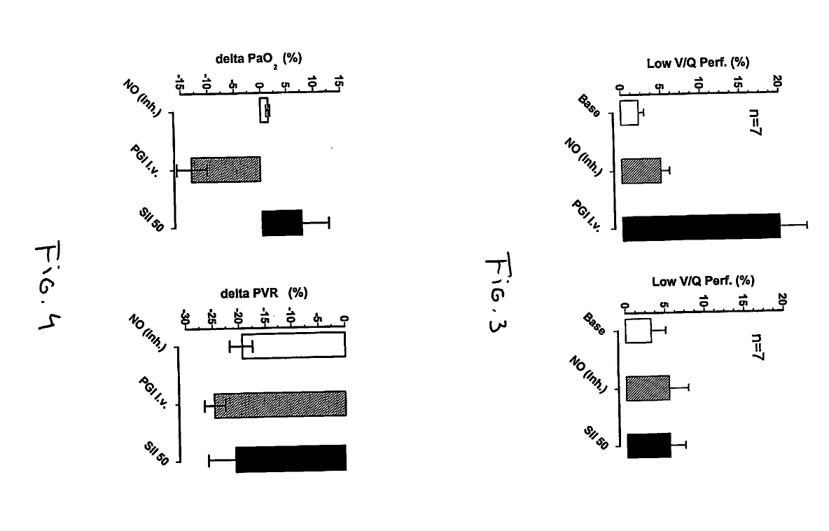

Secondary pulmonary hypertension, which is typical of these patients, was treated with PGI (intravenous), NO (inhaled) and with sildenafil (oral). The results on 7 patients show that perfusion of low-V/Q areas (V/Q<1) was increased slightly by NO (inhaled) but was greatly increased by PGI (intravenous). Sildenafil (oral) had the same effect as NO (inhaled) (FIG. 3). The arterial oxygen partial pressure was not changed by NO (inhaled), was reduced by 13% by PGI (intravenous) and increased with sildenafil (oral) (FIG. 4). The vascular resistance in the lungs (PVR) was, by contrast, reduced equally by 20-25% under the three conditions.

Consequently, it has been shown on patients with secondary pulmonary hypertension that all three vasodilators reduce pulmonary hypertension equally. Surprisingly, the influence of the vasodilators used on shunting varied widely. It was markedly increased by the systemic vasodilator PGI, whereas inhaled NO and the PDE5 inhibitor negligibly aggrevated the matching. The use of sildenafil and NO resulted in each case in a selective improvement in the pharmacological oxygenation, the value determined for sildenafil being improved by comparison with NO.

### Example 4

### Effects of Sildenafil on Patients with ILD (Interstitial Lung Disease)

7 patients with ILD underwent a Swan-Ganz catheter investigation with measurement of the ventilation/perfusion (V/Q) distribution (using the multiple inert gas elimination technique (MIGET)). After determination of the baseline parameters (haemodynamics and gas exchange), all the patients initially inhaled 20 ppm NO, followed by a second baseline period (10-15 min), and then PGI was infused (6 ng/kg bodyweight/min), again followed by a second baseline period (10-15 min) and then an oral dose of 50 mg of sildenafil was given (120-150 min follow-up). 5 of the 7 patients were controlled by continuous nasal oxygen therapy in order to reach an arterial oxygenation of >88%. The following parameters were measured under baseline conditions: mean pulmonary arterial pressure (mPAP) 39.6+/−2.8 mmHg and pulmonary vascular resistance index (PVRI) 1255+/−215 dyn*s*cm−5*m². MIGET demonstrated a blood flow through shunt areas (7.2+/−1.8%) and a large dead-space ventilation. Administration of NO, PGI and sildenafil led in each case to a marked reduction in the pulmonary vascular resistance. Whereas NO and sildenafil left the ventilation/perfusion distribution virtually unchanged on PGI infusion there was a considerable increase in the shunt perfusion (to 18%), resulting in a decrease in the arterial oxygen partial pressure during PGI infusion by 12.5% compared with the control investigation. The perfusion of normally distributed V/Q areas remained virtually unchanged.

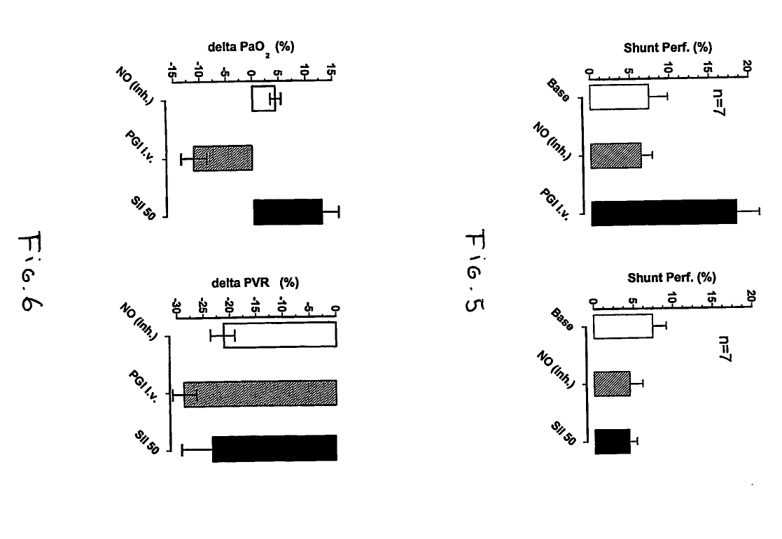

Patients with ILD displayed secondary pulmonary hypertension and were treated with vasodilators for this. The results on 7 patients show that NO (inhaled) and sildenafil (oral) had no effect on the markedly increased shunting in these patients. By contrast, PGI (intravenous) increased the lung areas from 7% to almost 20% and thus caused a deterioration in matching (FIG. 5). Corresponding to the matching, PGI reduced the O₂ saturation by 12%, whereas NO brought about an improvement of 5% and sildenafil one of 15% (FIG. 6). The secondary pulmonary vascular resistance (PVR) was distinctly reduced by about 25% with all three medications (FIG. 6).

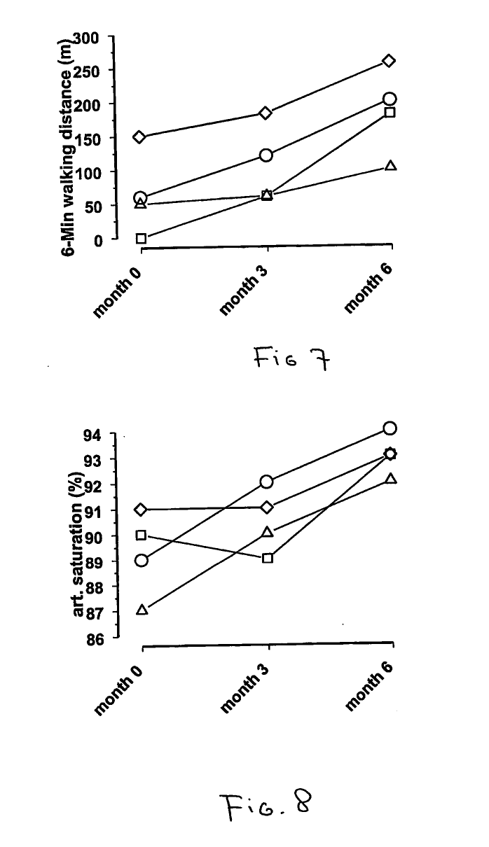

Improvements in the 6-min walking [Wijkstra et al., Thorax 1994, 49(5):468-72] (FIG. 7) and the corresponding measurements of the arterial O₂ saturation (FIG. 8) were followed on 4 patients during 6 months with 50 mg/d sildenafil (oral). The data show a marked improvement in the functional capacity of the investigated patients after administration of sildenafil over this time.

1. Use of PDE5 inhibitors for producing medicaments for the treatment of partial and global respiratory failure.

2. Use of PDE5 inhibitors in the treatment of partial and global respiratory failure.

3. Use of selective PDE5 inhibitors for producing medicaments for the treatment of respiratory failure in patients showing a mismatch of pulmonary ventilation and pulmonary perfusion.

4. Use according to claim 3, characterized in that patients with an exercise-dependent mismatch are treated.

5. Use of selective PDE5 inhibitors according to claim 3, characterized in that patients with an age-related mismatch are treated.

6. Use of selective PDE5 inhibitors according to claim 3, characterized in that patients with a pathologically caused mismatch are treated.

7. Use of selective PDE5 inhibitors according to any of claims 3 to 6, characterized in that patients with a mismatch of V/Q<0.1 are treated.

8. Use of selective PDE5 inhibitors according to claim 3, characterized in that COPD patients with a predominant bronchitic component are treated.

9. Use of selective PDE5 inhibitors according to claim 8, characterized in that COPD patients with a V/Q<0.1 are treated.

10. Use of selective PDE5 inhibitors according to claim 3, characterized in that COPD patients with an emphysematous component are treated.

11. Use of selective PDE5 inhibitors according to claim 10, characterized in that COPD patients with a V/Q>10 are treated.

12. Use of selective PDE5 inhibitors according to claim 3, characterized in that patients with orthopnoea are treated.

13. Use of selective PDE5 inhibitors according to claim 3, characterized in that patients with sleep apnoea are treated.

14. Use of selective PDE5 inhibitors according to claim 3, characterized in that the mismatch is therapy-induced.

15. Use of selective PDE5 inhibitors according to claim 14, characterized in that the mismatch is caused by administration of nonselectively vasodilating medicaments.

16. Use of selective PDE5 inhibitors according to claim 15, characterized in that a nonselectively vasodilating medicament is a nonselectively vasodilating antiobstructive agent.

17. Use of selective PDE5 inhibitors according to claim 16, characterized in that a nonselectively vasodilating antiobstructive agent is selected from the group consisting of endothelin antagonist, Ca channel blocker, ACE inhibitor, ATII antagonist and β blocker.

18. Use of PDE5 inhibitors for producing medicaments for the treatment of patients with muscular dysfunction caused by perfusion/demand mismatch.

19. Use according to any of claims 1 to 18, characterized in that the PDE5 inhibitor or the selective PDE5 inhibitor is an active ingredient which is selected from the group consisting of 3-ethyl-8-[2-(4-morpholinylmethyl)benzylamino]-2,3-dihydro-1H-imidazo[4,5-g]quinazoline-2-thione, 1-(2-chlorobenzyl)-3-isobutyryl-2-propylindole-6-carboxamide, 9-bromo-2-(3-hydroxypropoxy)-5-(3-pyridylmethyl)-4H-pyrido[3,2,1-[k]-carbazol-4-one, 4-(1,3-benzodioxol-5-ylmethylamino)-2-(1-imidazolyl)-6-methylthieno [2,3-pyrimidine, 6-(2-isopropyl-4,5,6,7-terahydropyrazolo[1,5-a]pyridin-3-yl)-5-methyl)-5-methyl-2, 3,4,5-tetrahydropyridazin-3-one, 5-(4-methylbenzyl)-3-(1-methyl-4-phenylbutyl)-3,6-dihydro-[1,2,3]triazolo[4,5-d]pyrimidin-7-one, 3-(1-methyl-4-phenylbutyl)-5-pyridin-4-ylmethyl-3,6-dihydro-[1,2,3]triazolo[4,5-d]pyrimidin-7-one, 5-(4-bromobenzyl)-3-(1-methyl-4-phenylbutyl)-3,6-dihydro-[1,2,3]triazolo[4,5-d]pyrimidin-7-one, 5-benzyl-3-(1-methyl-4-phenylbutyl)-3,6-dihydro-[1,2,3]triazolo[4,5-d]pyrimidin-7-one, 5-(3,4-dimethoxybenzyl)-3-(1-methyl-4-phenylbutyl)-3,6-dihydro-[1,2,3]triazolo-[4,5-d]pyrimidin-7-one, 5-(3,4-dichlorobenzyl)-3-(1-methyl-4-phenylbutyl)-3,6-dihydro-[1,2,3]triazolo[4,5-d]pyrimidin-7-one, 5-biphenyl-4-ylmethyl-3-(1-methyl-4-phenylbutyl)-3,6-dihydro-[1,2,3]triazolo[4,5-d]pyrimidin-7-one, 5-(4-aminobenzyl-3-(1-methyl-4-phenylbutyl)-3,6-dihydro-[1,2,3]triazolo[4,5-d]pyrimidin-7-one, 5-(hydroxyphenylmethyl)-3-(1-methyl-4-phenylbutyl)-3,6-dihydro-[1,2,3]triazolo-[4,5-d]pyrimidin-7-one, 5-benzo[1,3]dioxol-5-ylmethyl-3-[1-methyl-4-phenylbutyl]-3,6-dihydro-[1,2,3]triazolo[4,5-d]pyrimidin-7-one, N-4-[3-(1-methyl-4-phenylbutyl)-7-oxo-6,7-dihydro-3H-[1,2,3]triazolo-[4,5-d]pyrimidin-5-ylmethyl]phenylacetamide, 5-benzoyl-3-(1-methyl-4-phenylbutyl)-3,6-dihydro-[1,2,3]triazolo[4,5-d]-pyrimidin-7-one, 3-(1-methyl-4-phenylbutyl)-5-[4-(morpholine-4-sulphinyl) benzyl]-3,6-dihydro[1,2,3]triazolo[4,5-d]pyrimidin-7-one, 3-(1-methyl-4-phenylbutyl)-5-[3-(morpholine-4-sulphonyl) benzyl]-3,6-dihydro[1,2,3]triazolo[4,5-d]pyrimidin-7-one, N-methyl-4-[3-(1-methyl-4-phenylbutyl)-7-oxo-6,7-dihydro-3H-[1,2,3]-triazolo-[4,5-d]pyrimidin-5-ylmethyl]-benzenesulphonamide, N-(2-dimethylaminoethyl)-4-[3-(1-methyl-4-phenylbutyl)-7-oxo-6,7-dihydro-3H-[1,2,3]triazolo[4,5-d]pyrimidin-5-ylmethyl]benzenesulphonamide, N-(2-hydroxyethyl)-4-[3-(1-methyl-4-phenylbutyl)-7-oxo-6,7-dihydro-3H-[1,2,3]triazolo[4,5-d]pyrimidin-5-ylmethyl]benzenesulphonamide, ethyl 1-[3-[3-(1-methyl-4-phenylbutyl)-7-oxo-6,7-dihydro-3H-[1,2,3]-triazolo-[4,5-d]pyrimidin-5-ylmethyl]benzenesulphonyl]piperidinecarboxylate, 3(1-methyl-4-phenylbutyl)-5-[4-(4-methylpiperazin-1-sulphonyl) benzyl]-3,6-dihydro-[1,2,3]triazolo[4,5d]pyrimidin-7-one, 5-benzo[1,3]dioxol-5-ylmethyl-3-[1-ethyl-heptyl]-3,6-dihydro-[1,2,3]-triazolo[4,5-d]pyrimidin-7-one, 3-[1-(1-hydroxyethyl)-4-phenylbutyl]-5-[4-(morpholine-4-sulphonyl) benzyl]-3,6-dihydro-[1,2,3]triazolo[4,5-d]pyrimidin-7-one, 5-[6-fluoro-1-(phenylmethyl)-1H-indazol-3-yl]-2-furanmethanol, 1-benzyl-6-fluoro-3-[5-(hydroxymethyl)furan-2-yl]-1H-indazole, 2-(1H-imidazol-1-yl)-6-methoxy-4-(2-methoxyethylamino)quinazoline, 1-[[3-dihydro-8-oxo-1H-imidazo[4,5-g]quinazolin-6-yl)-4-propoxyphenyl]sulphonyl]-4-methylpiperazine, 4-(3-chloro-4-methoxybenzylamino)-1-(4-hydroxypiperidin-1-yl) phthalazine-6-carbonitrile, 1-[6-chloro-4-(3,4-methylendioxybenzylamino)quinazolin-2-yl]piperidin-4-carboxylic acid, (6R, 12aR)-6-(1,3-benzodioxol-5-yl)-2-methyl-1, 2,3,4,6,7,12,12a-octa-hydropyrazino[2′,1′:6,1]pyrido[3,4-b]indole-1,4-dione (tadalafil), (6R,12aR)-2,3,6,7,12,12a-hexahydro-2-methyl-6-(3,4-methylenedioxyphenyl)-pyrazino-[2′,1′:6,1]pyrido[3,4-b]indole-1,4-dione, 4-ethoxy-2-phenylcycloheptylimidazole, (6-bromo-3-methoxymethylimidazo [1,2-a]pyrazin-8-yl)methylamine, 8-[(phenylmethyl)thio]4-(1-morpholinyl)-2-(1-piperazinyl) pyrimidino[4,5-d]pyrimidine, (+)-cis-5-methyl-2-[4-(trifluoromethyl)benzyl]-3,4,5,6a,7,8,9-octahydrocyclopent [4,5]imidazo[2,1-b]purin-4-one, cis-2-hexyl-5-methyl-3,4,5,6a,7,8,9,9a-octahydrocyclopent [4,5imidazo[2,1-b]purin-4-one, 5-[2-ethoxy-5-(4-methyl-1-piperazinyl-sulphonyl) phenyl]-1-methyl-3-n-propyl-1,6-dihydro-7H-pyrazolo[4,3-d]pyrimidin-7-one (slidenafil), 1-[[3-(6,7-dihydro-1-methyl-7-oxo-3-propyl-1H-pyrazolo[4,3-d]pyrimidin-5-yl)-4-ethoxyphenyl]sulfonyl]-4-methylpiperazine, 2-(2-propoxyphenyl)purin-6(1H)-one, 2-(2-propoxyphenyl)-1,7-dihydro-5H-purin-6-one, methyl 2-(2-methylpyridin-4-ylmethyl)-1-oxo-8-(2-pyrimidinylmethoxy)-4-(3,4,5-trimethoxyphenyl)-1,2-dihydro-[2,7]naphthyridin-3-carboxylate, methyl 2-(4-aminophenyl)-1-oxo-7-(2-pyridinylmethoxy)-4-(3, 4,5-trimethoxyphenyl)-1,2-dihydroisoquinoline-3-carboxylate, 2-[2-ethoxy-5-(4-ethylpiperazin-1-ylsulfonyl) phenyl]-5-methyl-7-propylimidazo[5,1-f][1,2,4]triazin-4(3H)-one (vardenafil), 3,4-dihydro-6-[4-(3,4-dimethoxybenzoyl)-1-piperazinyl]-2-(1H)-quinolinone (vesnarinone), 1-cyclopentyl-3-methyl-6-(4-pyridyl) pyrazolo[3,4-d]pyrimidin-4(5H)-one, 1-cyclopentyl-6-(3-ethoxy-4-pyridinyl)-3-ethyl-1,7-dihydro-4H-pyrazolo[3,4-d]-pyrimidin-4-one, 6-o-propoxyphenyl-8-azapurin-6-one, 3,6-dihydro-5-(o-propoxyphenyl)-7H-v-triazolo[4,5-d]pyrimidin-7-one and 4-methyl-5-(4-pyridinyl)thiazole-2-carboxamide and the pharmacologically acceptable salts of these compounds.

20. Use according to any of claims 1 to 18, characterized in that the PDE5 inhibitor or the selective PDE5 inhibitor is an active ingredient selected from the group consisting of tadalafil, sildenafil, vardenafil and vesnarinone and the pharmacologically acceptable salts of these compounds.

21. Pharmaceutical preparation comprising at least one selective PDE5 inhibitor and at least one nonselectively vasodilating antiobstructive agent.

22. Pharmaceutical preparation according to claim 21 for the treatment of partial and global respiratory failure.

23. Pharmaceutical preparation according to claim 21 for the treatment of disorders selected from the group consisting of COPD, bronchial asthma, latent pulmonary hypertension, emphysema, combined ventilation disturbances and chronic left heart failure with pulmonary congestion.

24. Pharmaceutical preparation according to any of claims 21 to 23, characterized in that the selective PDE5 inhibitor is an active ingredient selected from the group consisting of 3-ethyl-8-[2-(4-morpholinylmethyl)benzylamino]-2,3-dihydro-1H-imidazo[4,5-g]quinazoline-2-thione, 1-(2-chlorobenzyl)-3-isobutyryl-2-propylindole-6-carboxamide, 9-bromo-2-(3-hydroxypropoxy)-5-(3-pyridylmethyl)-4H-pyrido[3,2,1-[k]-carbazol-4-one, 4-(1,3-benzodioxol-5-ylmethylamino)-2-(1-imidazolyl)-6-methylthieno [2,3-pyrimidine, 6-(2-isopropyl-4,5,6,7-terahydropyrazolo[1,5-a]pyridin-3-yl)-5-methyl)-5-methyl-2, 3,4,5-tetrahydropyridazin-3-one, 5-(4-methylbenzyl)-3-(1-methyl-4-phenylbutyl)-3,6-dihydro-[1,2,3]triazolo[4,5-d]pyrimidin-7-one, 3-(1-methyl-4-phenylbutyl)-5-pyridin-4-ylmethyl-3,6-dihydro-[1,2,3]triazolo[4,5-d]pyrimidin-7-one, 5-(4-bromobenzyl)-3-(1-methyl-4-phenylbutyl)-3,6-dihydro-[1,2,3]triazolo[4,5-d]pyrimidin-7-one, 5-benzyl-3-(1-methyl-4-phenylbutyl)-3,6-dihydro-[1,2,3]triazolo[4,5-d]pyrimidin-7-one, 5-(3,4-dimethoxybenzyl)-3-(1-methyl-4-phenylbutyl)-3,6-dihydro-[1,2,3]triazolo-[4,5-d]pyrimidin-7-one, 5-(3,4-dichlorobenzyl)-3-(1-methyl-4-phenylbutyl)-3,6-dihydro-[1,2,3]triazolo[4,5-d]pyrimidin-7-one, 5-biphenyl-4-ylmethyl-3-(1-methyl-4-phenylbutyl)-3,6-dihydro-[1,2,3]triazolo[4,5-d]pyrimidin-7-one, 5-(4-aminobenzyl-3-(1-methyl-4-phenylbutyl)-3,6-dihydro-[1,2,3]triazolo[4,5-d]pyrimidin-7-one, 5-(hydroxyphenylmethyl)-3-(1-methyl-4-phenylbutyl)-3,6-dihydro-[1,2,3]triazolo-[4,5-d]pyrimidin-7-one, 5-benzo[1,3]dioxol-5-ylmethyl-3-[1-methyl-4-phenylbutyl]-3,6-dihydro-[1,2,3]triazolo[4,5-d]pyrimidin-7-one, N-4-[3-(1-methyl-4-phenylbutyl)-7-oxo-6,7-dihydro-3H-[1,2,3]triazolo-[4,5-d]pyrimidin-5-ylmethyl]phenylacetamide, 5-benzoyl-3-(1-methyl-4-phenylbutyl)-3,6-dihydro-[1,2,3]triazolo[4,5-d]-pyrimidin-7-one, 3-(1-methyl-4-phenylbutyl)-5-[4-(morpholine-4-sulphinyl) benzyl]-3,6-dihydro[1,2,3]triazolo[4,5-d]pyrimidin-7-one, 3-(1-methyl-4-phenylbutyl)-5-[3-(morpholine-4-sulphonyl) benzyl]-3,6-dihydro[1,2,3]triazolo[4,5-d]pyrimidin-7-one, N-methyl-4-[3-(1-methyl-4-phenylbutyl)-7-oxo-6,7-dihydro-3H-[1,2,3]-triazolo-[4,5-d]pyrimidin-5-ylmethyl]-benzenesulphonamide, N-(2-dimethylaminoethyl)-4-[3-(1-methyl-4-phenylbutyl)-7-oxo-6,7-dihydro-3H-[1,2,3]triazolo[4,5-d]pyrimidin-5-ylmethyl]benzenesulphonamide, N-(2-hydroxyethyl)-4-[3-(1-methyl-4-phenylbutyl)-7-oxo-6,7-dihydro-3H-[1,2,3]triazolo[4,5-d]pyrimidin-5-ylmethyl]benzenesulphonamide, ethyl 1-[3-[3-(1-methyl-4-phenylbutyl)-7-oxo-6,7-dihydro-3H-[1,2,3]-triazolo-[4,5-d]pyrimidin-5-ylmethyl]benzenesulphonyl]piperidinecarboxylate, 3(1-methyl-4-phenylbutyl)-5-[4-(4-methylpiperazin-1-sulphonyl) benzyl]-3,6-dihydro-[1,2,3]triazolo[4,5d]pyrimidin-7-one, 5-benzo[1,3]dioxol-5-ylmethyl-3-[1-ethyl-heptyl]-3,6-dihydro-[1,2,3]-triazolo[4,5-d]pyrimidin-7-one, 3-[1-(1-hydroxyethyl)-4-phenylbutyl]-5-[4-(morpholine-4-sulphonyl) benzyl]-3,6-dihydro-[1,2,3]triazolo[4,5-d]pyrimidin-7-one, 5-[6-fluoro-1-(phenylmethyl)-1H-indazol-3-yl]-2-furanmethanol, 1-benzyl-6-fluoro-3-[5-(hydroxymethyl)furan-2-yl]-1H-indazole, 2-(1H-imidazol-1-yl)-6-methoxy-4-(2-methoxyethylamino)quinazoline, 1-[[3-dihydro-8-oxo-1H-imidazo[4,5-g]quinazolin-6-yl)-4-propoxyphenyl]sulphonyl]-4-methylpiperazine, 4-(3-chloro-4-methoxybenzylamino)-1-(4-hydroxypiperidin-1-yl) phthalazine-6-carbonitrile, 1-[6-chloro-4-(3,4-methylendioxybenzylamino)quinazolin-2-yl]piperidin-4-carboxylic acid, (6R, 12aR)-6-(1,3-benzodioxol-5-yl)-2-methyl-1, 2,3,4,6,7,12,12a-octa-hydropyrazino[2′,1′:6,1]pyrido[3,4-b]indole-1,4-dione (tadalafil), (6R,12aR)-2,3,6,7,12,12a-hexahydro-2-methyl-6-(3,4-methylenedioxyphenyl)-pyrazino-[2′,1′:6,1]pyrido[3,4-b]indole-1,4-dione, 4-ethoxy-2-phenylcycloheptylimidazole, (6-bromo-3-methoxymethylimidazo [1,2-a]pyrazin-8-yl)methylamine, 8-[(phenylmethyl)thio]4-(1-morpholinyl)-2-(1-piperazinyl) pyrimidino[4,5-d]pyrimidine, (+)-cis-5-methyl-2-[4-(trifluoromethyl)benzyl]-3,4,5,6a,7,8,9-octahydrocyclopent [4,5]imidazo[2,1-b]purin-4-one, cis-2-hexyl-5-methyl-3,4,5,6a,7,8,9,9a-octahydrocyclopent [4,5imidazo[2,1-b]purin-4-one, 5-[2-ethoxy-5-(4-methyl-1-piperazinyl-sulphonyl) phenyl]-1-methyl-3-n-propyl-1,6-dihydro-7H-pyrazolo[4,3-d]pyrimidin-7-one (slidenafil), 1-[[3-(6,7-dihydro-1-methyl-7-oxo-3-propyl-1H-pyrazolo[4,3-d]pyrimidin-5-yl)-4-ethoxyphenyl]sulfonyl]-4-methylpiperazine, 2-(2-propoxyphenyl)purin-6(1H)-one, 2-(2-propoxyphenyl)-1,7-dihydro-5H-purin-6-one, methyl 2-(2-methylpyridin-4-ylmethyl)-1-oxo-8-(2-pyrimidinylmethoxy)-4-(3,4,5-trimethoxyphenyl)-1,2-dihydro-[2,7]naphthyridin-3-carboxylate, methyl 2-(4-aminophenyl)-1-oxo-7-(2-pyridinylmethoxy)-4-(3, 4,5-trimethoxyphenyl)-1,2-dihydroisoquinoline-3-carboxylate, 2-[2-ethoxy-5-(4-ethylpiperazin-1-ylsulfonyl) phenyl]-5-methyl-7-propylimidazo[5,1-f][1,2,4]triazin-4(3H)-one (vardenafil), 3,4-dihydro-6-[4-(3,4-dimethoxybenzoyl)-1-piperazinyl]-2-(1H)-quinolinone (vesnarinone), 1-cyclopentyl-3-methyl-6-(4-pyridyl) pyrazolo[3,4-d]pyrimidin-4(5H)-one, 1-cyclopentyl-6-(3-ethoxy-4-pyridinyl)-3-ethyl-1,7-dihydro-4H-pyrazolo[3,4-d]-pyrimidin-4-one, 6-o-propoxyphenyl-8-azapurin-6-one, 3,6-dihydro-5-(o-propoxyphenyl)-7H-v-triazolo[4,5-d]pyrimidin-7-one and 4-methyl-5-(4-pyridinyl)thiazole-2-carboxamide and the pharmacologically acceptable salts of these compounds.

25. Pharmaceutical preparation according to any of claims 21 to 23, characterized in that the selective PDE5 inhibitor is an active ingredient selected from the group consisting of tadalafil, sildenafil, vardenafil and vesnarinone and the pharmacologically acceptable salts of these compounds.

26. Commercial product consisting of a conventional secondary packaging, of a primary packaging containing the medicament and, if desired, of a package insert, where the medicament is used for the treatment of partial and global respiratory failure, the suitability of the medicament for the treatment of partial and global respiratory failure is indicated on the secondary packaging and/or on the package insert of the commercial product, and the medicament comprises an active ingredient from the class of PDE5 inhibitors.

27. Ready-to-use medicament comprising a PDE5 inhibitor and an indication that this medicament can be employed for the treatment of partial and global respiratory failure.

28. A method of treating partial and global respiratory failure in a human in need thereof comprising the step of administering to said human a therapeutically effective amount of a PDE5 inhibitor.

29. A method of treating respiratory failure in a human showing a mismatch of pulmonary ventilation and pulmonary perfusion comprising the steps of administration to said human in need a therapeutically effective amount of a selective PDE5 inhibitor.

30. The method according to claim 29, wherein the human in need has an exercise-dependent mismatch.

31. The method according to claim 29, wherein the human in need has an age-related mismatch.

32. The method according to claim 29, wherein the human in need has a pathologically caused mismatch.

33. The method according to claim 29 to 32, wherein the human in need has a mismatch of V/Q<0.1.

34. The method according to claim 29, wherein the human in need is a COPD patient with a predominant bronchitis component.

35. The method according to claim 34, wherein the human in need is a COPD patient with a V/Q<0.1.

36. The method according to claim 29, wherein the human in need is a COPD patient with an emphysematous component.

37. The method according to claim 36, wherein the human in need is a COPD patient with a V/Q>10.

38. A method according to claim 29, wherein the human in need has orthopnoea.

39. A method according to claim 29, wherein the human in need has sleep apnoea.

40. The method according to claim 29, wherein the human in need has a therapy-induced mismatch.

41. The method according to claim 40, wherein the human in need has a mismatch caused by administration of nonselectively vasodilating medicaments.

42. The method according to claim 41, wherein the nonselectively vasodilating medicament is a nonselectively vasodilating antiobstructive agent.

43. The method according to claim 42, wherein the nonselectively vasodilating antiobstructive agent is selected from the group consisting of endothelin antagonist, Ca channel blocker, ACE inhibitor, ATII antagonist and β blocker.

44. A method of treating muscular dysfunction in a human showing a perfusion/demand mismatch comprising the step of administering to said human a therapeutically effective amount of a PDE5 inhibitor.

45. The method according to one of the claims 28 to 44, characterized in that the PDE5 inhibitor or the selective PDE5 inhibitor is an active ingredient which is selected from the group consisting of 3-ethyl-8-[2-(4-morpholinylmethyl)benzylamino]-2,3-dihydro-1H-imidazo[4,5-g]quinazoline-2-thione, 1-(2-chlorobenzyl)-3-isobutyryl-2-propylindole-6-carboxamide, 9-bromo-2-(3-hydroxypropoxy)-5-(3-pyridylmethyl)-4H-pyrido[3,2,1-[k]-carbazol-4-one, 4-(1,3-benzodioxol-5-ylmethylamino)-2-(1-imidazolyl)-6-methylthieno [2,3-pyrimidine, 6-(2-isopropyl-4,5,6,7-terahydropyrazolo[1,5-a]pyridin-3-yl)-5-methyl)-5-methyl-2, 3,4,5-tetrahydropyridazin-3-one, 5-(4-methylbenzyl)-3-(1-methyl-4-phenylbutyl)-3,6-dihydro-[1,2,3]triazolo[4,5-d]pyrimidin-7-one, 3-(1-methyl-4-phenylbutyl)-5-pyridin-4-ylmethyl-3,6-dihydro-[1,2,3]triazolo[4,5-d]pyrimidin-7-one, 5-(4-bromobenzyl)-3-(1-methyl-4-phenylbutyl)-3,6-dihydro-[1,2,3]triazolo[4,5-d]pyrimidin-7-one, 5-benzyl-3-(1-methyl-4-phenylbutyl)-3,6-dihydro-[1,2,3]triazolo[4,5-d]pyrimidin-7-one, 5-(3,4-dimethoxybenzyl)-3-(1-methyl-4-phenylbutyl)-3,6-dihydro-[1,2,3]triazolo-[4,5-d]pyrimidin-7-one, 5-(3,4-dichlorobenzyl)-3-(1-methyl-4-phenylbutyl)-3,6-dihydro-[1,2,3]triazolo[4,5-d]pyrimidin-7-one, 5-biphenyl-4-ylmethyl-3-(1-methyl-4-phenylbutyl)-3,6-dihydro-[1,2,3]triazolo[4,5-d]pyrimidin-7-one, 5-(4-aminobenzyl-3-(1-methyl-4-phenylbutyl)-3,6-dihydro-[1,2,3]triazolo[4,5-d]pyrimidin-7-one, 5-(hydroxyphenylmethyl)-3-(1-methyl-4-phenylbutyl)-3,6-dihydro-[1,2,3]triazolo-[4,5-d]pyrimidin-7-one, 5-benzo[1,3]dioxol-5-ylmethyl-3-[1-methyl-4-phenylbutyl]-3,6-dihydro-[1,2,3]triazolo[4,5-d]pyrimidin-7-one, N-4-[3-(1-methyl-4-phenylbutyl)-7-oxo-6,7-dihydro-3H-[1,2,3]triazolo-[4,5-d]pyrimidin-5-ylmethyl]phenylacetamide, 5-benzoyl-3-(1-methyl-4-phenylbutyl)-3,6-dihydro-[1,2,3]triazolo[4,5-d]-pyrimidin-7-one, 3-(1-methyl-4-phenylbutyl)-5-[4-(morpholine-4-sulphinyl) benzyl]-3,6-dihydro[1,2,3]triazolo[4,5-d]pyrimidin-7-one, 3-(1-methyl-4-phenylbutyl)-5-[3-(morpholine-4-sulphonyl) benzyl]-3,6-dihydro[1,2,3]triazolo[4,5-d]pyrimidin-7-one, N-methyl-4-[3-(1-methyl-4-phenylbutyl)-7-oxo-6,7-dihydro-3H-[1,2,3]-triazolo-[4,5-d]pyrimidin-5-ylmethyl]-benzenesulphonamide, N-(2-dimethylaminoethyl)-4-[3-(1-methyl-4-phenylbutyl)-7-oxo-6,7-dihydro-3H-[1,2,3]triazolo[4,5-d]pyrimidin-5-ylmethyl]benzenesulphonamide, N-(2-hydroxyethyl)-4-[3-(1-methyl-4-phenylbutyl)-7-oxo-6,7-dihydro-3H-[1,2,3]triazolo[4,5-d]pyrimidin-5-ylmethyl]benzenesulphonamide, ethyl 1-[3-[3-(1-methyl-4-phenylbutyl)-7-oxo-6,7-dihydro-3H-[1,2,3]-triazolo-[4,5-d]pyrimidin-5-ylmethyl]benzenesulphonyl]piperidinecarboxylate, 3(1-methyl-4-phenylbutyl)-5-[4-(4-methylpiperazin-1-sulphonyl) benzyl]-3,6-dihydro-[1,2,3]triazolo[4,5d]pyrimidin-7-one, 5-benzo[1,3]dioxol-5-ylmethyl-3-[1-ethyl-heptyl]-3,6-dihydro-[1,2,3]-triazolo[4,5-d]pyrimidin-7-one, 3-[1-(1-hydroxyethyl)-4-phenylbutyl]-5-[4-(morpholine-4-sulphonyl) benzyl]-3,6-dihydro-[1,2,3]triazolo[4,5-d]pyrimidin-7-one, 5-[6-fluoro-1-(phenylmethyl)-1H-indazol-3-yl]-2-furanmethanol, 1-benzyl-6-fluoro-3-[5-(hydroxymethyl)furan-2-yl]-1H-indazole, 2-(1H-imidazol-1-yl)-6-methoxy-4-(2-methoxyethylamino)quinazoline, 1-[[3-dihydro-8-oxo-1H-imidazo[4,5-g]quinazolin-6-yl)-4-propoxyphenyl]sulphonyl]-4-methylpiperazine, 4-(3-chloro-4-methoxybenzylamino)-1-(4-hydroxypiperidin-1-yl) phthalazine-6-carbonitrile, 1-[6-chloro-4-(3,4-methylendioxybenzylamino)quinazolin-2-yl]piperidin-4-carboxylic acid, (6R, 12aR)-6-(1,3-benzodioxol-5-yl)-2-methyl-1, 2,3,4,6,7,12,12a-octa-hydropyrazino[2′,1′:6,1]pyrido[3,4-b]indole-1,4-dione (tadalafil), (6R,12aR)-2,3,6,7,12,12a-hexahydro-2-methyl-6-(3,4-methylenedioxyphenyl)-pyrazino-[2′,1′:6,1]pyrido[3,4-b]indole-1,4-dione, 4-ethoxy-2-phenylcycloheptylimidazole, (6-bromo-3-methoxymethylimidazo [1,2-a]pyrazin-8-yl)methylamine, 8-[(phenylmethyl)thio]4-(1-morpholinyl)-2-(1-piperazinyl) pyrimidino[4,5-d]pyrimidine, (+)-cis-5-methyl-2-[4-(trifluoromethyl)benzyl]-3,4,5,6a,7,8,9-octahydrocyclopent [4,5]imidazo[2,1-b]purin-4-one, cis-2-hexyl-5-methyl-3,4,5,6a,7,8,9,9a-octahydrocyclopent [4,5imidazo[2,1-b]purin-4-one, 5-[2-ethoxy-5-(4-methyl-1-piperazinyl-sulphonyl) phenyl]-1-methyl-3-n-propyl-1,6-dihydro-7H-pyrazolo[4,3-d]pyrimidin-7-one (slidenafil), 1-[[3-(6,7-dihydro-1-methyl-7-oxo-3-propyl-1H-pyrazolo[4,3-d]pyrimidin-5-yl)-4-ethoxyphenyl]sulfonyl]-4-methylpiperazine, 2-(2-propoxyphenyl)purin-6(1H)-one, 2-(2-propoxyphenyl)-1,7-dihydro-5H-purin-6-one, methyl 2-(2-methylpyridin-4-ylmethyl)-1-oxo-8-(2-pyrimidinylmethoxy)-4-(3,4,5-trimethoxyphenyl)-1,2-dihydro-[2,7]naphthyridin-3-carboxylate, methyl 2-(4-aminophenyl)-1-oxo-7-(2-pyridinylmethoxy)-4-(3, 4,5-trimethoxyphenyl)-1,2-dihydroisoquinoline-3-carboxylate, 2-[2-ethoxy-5-(4-ethylpiperazin-1-ylsulfonyl) phenyl]-5-methyl-7-propylimidazo[5,1-f][1,2,4]triazin-4(3H)-one (vardenafil), 3,4-dihydro-6-[4-(3,4-dimethoxybenzoyl)-1-piperazinyl]-2-(1H)-quinolinone (vesnarinone), 1-cyclopentyl-3-methyl-6-(4-pyridyl) pyrazolo[3,4-d]pyrimidin-4(5H)-one, 1-cyclopentyl-6-(3-ethoxy-4-pyridinyl)-3-ethyl-1,7-dihydro-4H-pyrazolo[3,4-d]-pyrimidin-4-one, 6-o-propoxyphenyl-8-azapurin-6-one, 3,6-dihydro-5-(o-propoxyphenyl)-7H-v-triazolo[4,5-d]pyrimidin-7-one and 4-methyl-5-(4-pyridinyl)thiazole-2-carboxamide and the pharmacologically acceptable salts of these compounds.

46. The method according to one of the claims 28 to 44, characterized in that the PDE5 inhibitor or the selective PDE5 inhibitor is an active ingredient selected from the group consisting of tadalafil, sildenafil, vardenafil and vesnarinone and the pharmacologically acceptable salts of these compounds.
